(19) United States
(12) Patent Application Publication
(10) Pub. No.: US 2026/0287570 A1
(43) Pub. Date: Sep. 24, 2026

(54) METHOD, SYSTEM AND KIT FOR DETECTING PERFLUOROALKYL AND POLYFLUOROALKYL (PFAS) COMPOUNDS

(71) Applicant: The United States of America, as represented by the Secretary of the Navy, Arlington, VA (US)

(72) Inventors: Lewis Hsu, Aiea, HI (US); Cody K. Hayashi, Waipahu, HI (US); Mario Malfavon, San Diego, CA (US); Channing E. Bolt, San Diego, CA (US); Nicholas T. Hayman, San Diego, CA (US); Robert D. George, Chula Vista, CA (US)

(73) Assignee: The United States of America, as represented by the Secretary of the Navy, Arlington, VA (US)

(21) Appl. No.: 19/031,744

(22) Filed: Jan. 18, 2025

(51) Int. Cl. G01N 33/18; G01N 1/34; G01N 27/30; G01N 27/42

(52) U.S. Cl. CPC G01N 33/1826; G01N 1/34; G01N 27/30; G01N 27/42

(57) ABSTRACT

The present invention includes systems, methods and field portable kits for detecting per- and polyfluorinated compounds (PFAS) in wastewater. The invention includes removal of oil and/or grease from the wastewater using electrocoagulation techniques prior to PFAS detection, if necessary. The invention employs low cost, disposable screen-printed electrodes for detecting PFAS. The invention provides the ability to perform detection of analytes such as PFAS in a liquid medium with a disposable, portable, and rugged system at remote locations including onboard marine vessels.

### FEDERALLY SPONSORED RESEARCH AND DEVELOPMENT

The United States Government has ownership rights in this invention. Licensing and technical inquiries may be directed to the Office of Research and Technical Applications, Naval Information Warfare Center Pacific, Code 72120, San Diego, CA, 92152; voice: (619) 553-5118; email: NIWC_Pacific_T2@navy.mil. Reference Navy Case Number 212015.

### BACKGROUND OF THE INVENTION

Field of the Invention: The present invention relates generally to systems and methods for wastewater treatment. More particularly, the present invention relates to systems and methods for determining the presence of contaminants in wastewater. Still more particularly, the present invention is directed to methods, systems and kits for detecting perfluoroalkyl and polyfluoroalkyl (PFAS) compounds in wastewater.

Description of Related Art: Perfluoroalkyl and polyfluoroalkyl substances, which are also known as per- and polyfluorinated compounds and are collectively referred to herein as “PFAS”. The term “bilge water” is defined herein as water emanating from the bilge of a marine vessel. The term “oily wastewater” or “OWW” is defined herein as wastewater that also includes oil and/or grease. OWW may be visually distinguishable over non-oily wastewater by the appearance of a sheen caused by a thin film of oil on the water's surface, causing light to reflect differently due to the difference in the refractive index between oil and water, resulting in an iridescent or shimmering appearance, often described as a “sheen.” Wastewater may or may not contain PFAS which require specialized treatment. For this reason, systems and methods of detecting PFAS in wastewater are particularly useful.

During marine vessel operation, oily wastewater (OWW) often accumulates in the bilge compartment and commingles with a variety of contaminants. The engine rooms and machinery aboard marine vessels generate OWW, also known as bilge water. Vessels are generally not allowed to discharge OWW to harbors or marinas when near shore or in port and must coordinate disposal with shore-side facilities. The most common disposal method is to send the oily wastewater to a pre-treatment facility to remove the oily fraction prior to disposal at a wastewater treatment plant.

### Oily Wastewater

OWW is a complex matrix that can contain several contaminants that make them a threat to environmental and human health. These include oils, fuels, detergents, heavy metals, other organic pollutants, etc., see, e.g., Han, Meiling, Jin Zhang, Wen Chu, Jiahao Chen, and Gongfu Zhou, “Research Progress and Prospects of Marine Oily Wastewater Treatment: A Review.” Water 11 (12): 2517, 2019; and Church, Jared, Jeffrey Lundin, Daniela Diaz, Dianne Mercado, Marjorie Wilner, Woo Hyoung Lee, and Danielle Paynter, “Identification and characterization of bilgewater emulsions”, Science of The Total Environment 691:981-995, 2019. As such, OWW is difficult to treat. Multiple contaminants frequently require several different types of treatment and interactions between the contaminants can sharply reduce the effectiveness of these treatment technologies. From Applicant's perspective, bilgewater may be the largest source of OWW generated by afloat forces.

One of the most challenging aspects of OWW in terms of chemical analyses is the oil/water emulsions. Oil/water emulsions form in bilgewater due to mechanical mixing energy from waves, see, e.g., Eskandarloo, Hamed, Michael J Selig, and Alireza Abbaspourrad, “In situ H₂O₂ generation for de-emulsification of fine stable bilge water emulsions”, Chemical Engineering Journal 434-442, 2018. Oil/water emulsions may also form in bilgewater from surfactant stabilization due to surfactants commonly found in detergents, cleaning agents, aqueous film forming foams, etc., see, e.g., Church, Jared, Jeffrey Lundin, Daniela Diaz, Dianne Mercado, Marjorie Wilner, Woo Hyoung Lee, and Danielle Paynter, “Identification and characterization of bilgewater emulsions”, Science of The Total Environment 691:981-995, 2019. Emulsified oil, defined as oil droplets <20 μm, are difficult to remove as they are neutrally buoyant (i.e., cannot use gravimetric separation) and highly stable. These oil emulsifications can lead to interferences for analytical chemistry, resulting in difficulty to accurately determine PFAS concentrations.

Typically, overtime, these oil/water emulsions break down due to thermodynamic instability. As may be expected, methods to separate oil from water in OWW try to take advantage of these processes. These various processes include creaming/sedimentation, flocculation, coalescence, and Oswald ripening see, e.g., Church, et al., supra. Flocculation will eventually lead to coalescence of the oil on the surface or bottom of the solution, depending on the oil or floc density.

### Handling of OWW

During vessel operation, bilge water generally accumulates in the bottom of a ship, or bilge compartment, and commingles with a variety of contaminants. Bilge water may be pumped into a dedicated holding tank using a bilge pump. Bilge water may contain contaminants from machinery spaces, internal drainage systems, sludge tanks, and other sources. It will be understood that bilge water is wastewater and may also be OWW, i.e., mixture of water, oil, grease, sludge, and other chemicals.

Marine vessels are allowed to discharge bilge water at sea, but only if it has been properly treated through an oil-water separator (OWS) to ensure the oil content is below a specific limit, typically 15 parts per million (ppm), as stipulated by international regulations such as the International Convention for the Prevention of Pollution from Ships (MARPOL). Thus, vessels can only discharge “clean” bilge water into the ocean. On ships without OWS systems, untreated bilgewater is held for transfer to a shore treatment facility.

As part of firefighting activities, OWW may contain trace amounts of aqueous film forming foams (AFFF). Historically, AFFF formulations have used long chain fluorocarbon compounds due to their inert nature and ability to form oxygen blocking films on flammable liquid surfaces. Recent reports or their recalcitrance in the environment and potentially harmful health effects have made responsible disposal a primary concern in order to prevent future exposure of even trace amounts of the chemical. In certain foreign ports, detection of a specific fluorocarbon, perfluorooctanesulfonic acid (PFOS), above specified levels (typically in the low ng/L levels) require special disposal and handling. PFOS, which has the chemical formula CF₃(CF₂)₇SO₃, is a perfluorinated compound specifically and a compound belonging to the class of PFAS generally. PFOS and other PFAS substances are found in many commercial products and industrial processes.

Utilization of oily wastewater collection systems at shore facilities are required for Applicant's (US Navy) vessels when moored pierside or returning to homeport. While in state and federal waters (less than 12 nautical miles from shore), collected oily wastewater is often processed through specialized shore systems (i.e., the bilge and oily wastewater treatment system) to separate oil and grease fractions from the bulk wastewater originating from vessels. In certain situations, chemical contaminants such as metals or organics may be found within discharged oily wastewater. Such metals and organic constituents are generally easily treated in industrial wastewater facilities. However, bilge water may also include environmentally controlled contaminants such as PFAS including PFOS are not easily treated with conventional wastewater facilities and may require additional treatment. PFAS are currently of special concern to regulatory agencies due to their widespread prevalence in the environment and suspected human health effects, even at low concentrations.

### Coagulation-Flocculation

The use of coagulation-flocculation for oil and grease removal in wastewater is well-known in conventional wastewater treatment. Such conventional techniques generally require the addition of a metal in a chemical form, generally alum or ferric chloride, to the wastewater, followed by mixing to encourage metal hydroxide formation and subsequent precipitation. Precipitates are encouraged to aggregate into flocs that are removed through settling or surface skimming. See, e.g., Zhao, Chuanliang, Junyuan Zhou, Yi Yan, Liwei Yang, Guohua Xing, Huanyu Li, Pei Wu, Mingyuan Wang, and Huaili Zheng, “Application of coagulation/flocculation in oily wastewater treatment: A review.” Science of the Total Environment 765:142795, 2021; and also, Abuhasel, Khaled, Mohamed Kchaou, Mohammed Alquraish, Yamuna Munusamy, and Yong Tzyy Jeng, “OilyWastewater Treatment: Overview of Conventional and Modern Methods, Challenges, and Future Opportunities,”, Water 13:980, 2021.

### Testing OWW for Contaminants

The US Environmental Protection Agency (EPA) prescribes a couple methods for measuring PFAS, namely EPA 537 and a more recent EPA 1633. Both of these EPA methods require advanced instrumentation, namely a liquid chromatograph equipped with tandem mass spectrometers, which is generally only available in analytical laboratories and research institutions. In addition, due to challenging quality assurance and quality control requirements, few labs in the contiguous United States (CONUS) are currently certified for these methods. This limited testing capability and distance from foreign home ports has resulted in delays in confirming the absence of PFAS in OWW collected for disposal at shore facilities outside the contiguous United States (OCONUS).

Most published methodologies for detecting PFAS not requiring a liquid chromatography instrument generally fall within two categories: (1) chemical/optical detection method and (2) electrochemical analysis, see, e.g., Ryu, Heejeong, Baikum Li, Sylvain De Guise, Jeffrey Mccutcheon, Yu Lei, “Recent progress in the detection of emerging contaminants PFASs”, Journal of Hazardous Materials 408:124, 2021. Chemical/optical approaches to PFAS detection, for example, generally rely upon interaction of PFAS with a detection chemical that changes color or fluorescence. Colorimetric methods of detection are advantageous since they are typically easy to use and have a rapid response. Recent studies developing colorimetric sensing methods for PFAS have reported on the use of nanomaterials as the sensing material, see, e.g., Garg, Shafali, Pankaj Kumar, George Greene, Vandana Mishra, Dror Avisar, Radhey Sharma, and Ludovic Dumee, “Nano-enabled sensing of per-/poly-fluoroalkyl substances (PFAS) from aqueous systems—A review”, Journal of Environmental Management 308:1146, 2022. Nanomaterial-related sensing methods have been reported with detection levels approaching parts per billion (μg/L), see, e.g., Fang, Cheng, Xian Zhang, Zhaomin Dong, Liang Wang, Mallavarapu Megharaj, and Ravi Naidu, “Smartphone app-based/portable sensor for the detection of fluoro-surfactant PFOA”, Chemosphere 191:381-88, 2018; Niu, Hongyun, Saihua Wang, Zhen Zhou, Yurong Ma, Xunfeng Ma, and Yaqi Cai, “Sensitive Colorimetric Visualization of Perfluorinated Compounds Using Poly(ethylene glycol) and Perfluorinated Thiols Modified Gold Nanoparticles”, Analytical Chemistry 86 (9): 4170-417, 2014; Cong, Yan-bin, Yong-hong Zheng, Li Zheng, Fei Wu, and Ke-jun Tan, “Colorimetric Assay of Perfluorooctanesulfonate Based on Gold Nanoparticles”, Spectroscopy and Spectral Analysis 35 (1): 189-19, 2015. Similarly, use of dyes or other color compounds in sensing technology has been reported to yield detection levels at parts per million (mg/L) or parts per billion levels, see, e.g., Menger, Ruth, Josianne Beck, Thomas Borch, and Charles Henry, “Colorimetric Paper-Based Analytical Device for Perfluorooctanesulfonate Detection”, ACS EST Water 4:565-57, 2022; and He, Jincon, Peipei Qiu, Jiayi Song, Shiyun Zhang, and Yan Bai, “A resonance Rayleigh scattering and colorimetric dual-channel sensor for sensitive detection of perfluorooctane sulfonate based on toluidine blue”, Analytical and Bioanalytical Chemistry 412:5329-533, 2020. These methods may also use a preconcentration step, solid phase extraction, to enhance detection limits. However, these detection limits are still well above the limits set forth for regulatory action.

Fluorescence-based techniques also focus on utilizing nanomaterials to report concentrations based on interaction with PFAS to deactivate or activate the fluorescent phenomena. Strategies to deactivate fluorescence have utilized naturally fluorescent materials such as carbon dots, quantum dots, or dyes. Reports using this “switch off” strategy of detection have been relatively sensitive, with limits of detection reported in the parts per billion and part per trillion levels, see, e.g., Feng, Hui, Niya Wang, Thanh Thuy Tran. T, Lijuan Yuan, Jiezhen Li, and Qingyun Cai, “Surface molecular imprinting on dye-(NH2)-SiO2 NPs for specific recognition and direct fluorescent quantification of perfluorooctane sulfonate”, Sensors and Actuators B: Chemical 195:266-27, 2014; and Zhang, Qiaojuan, Mengyu Liao, Keren Xiao, Kangyuan Zhuang, Weilian Zheng, and Zhiyi Yao, “A water-soluble fluorescence probe based on perylene diimide for rapid and selective detection of perfluorooctane sulfonate in 100% aqueous media”, Sensors and Actuators B: Chemical 350:1308, 2022. Alternatively, a “switch on” approach has also been reported where interaction of PFOS with a reporter appears to enhance fluorescence emissions with similar limits of detection, see, e.g., Cheng, Zhen, Hongcen Dong, Jiaman Liang, Fang Zhang, Xianping Chen, Lingling Du, and Kejun Tan, “Highly selective fluorescent visual detection of perfluorooctane sulfonate via blue fluorescent carbon dots and berberine chloride hydrate”, Spectrochimica Acta Part A: Molecular and Biomolecular Spectroscopy 207:262-26, 2019; and Cheng, Zhen, Lingling Du, Panpan Zhu, Qian Chen, and Kejun Tan, “An erythrosin B-based “turn on” fluorescent sensor for detecting perfluorooctane sulfonate and perfluorooctanoic acid in environmental water samples”, Spectrochimica Acta Part A: Molecular and Biomolecular Spectroscopy 201:281-28, 2018. Fluorescence-based biosensors for PFOS are not as prevalent as chemical approaches, however one bacterial sensor has been reported to detect PFOS levels down to parts per trillion levels, see, e.g., Sunantha, Ganesan, and Namasivayam Vasudevan, “A method for detecting perfluorooctanoic acid and perfluorooctane sulfonate in water samples using genetically engineered bacterial biosensor”, Science of The Total Environment 759:1435, 2021. These optical methods usually require specialized chemicals which can require special handling requirements and optical measurement capabilities that may be difficult to replicate in small or portable applications, see, e.g., Shin, Young-Ho, M. Teresa Gutierrez-Wing, Jin-Woo Choi, “Recent Progress in Portable Fluorescence Sensors”, Journal of The Electrochemical Society 168:0175, 2021.

Electrochemical analyses rely on the interaction of PFAS with an electrode surface during programmed changes in voltage or current. There are two approaches: voltammetry or impedance. In voltametric approaches, a current or voltage signal is monitored as electrical signals are used to interrogate an electrode surface. Several different electrochemical tests can be performed. Differential pulse voltammetry has been utilized with a molecularly imprinted polymer (MIP) modified electrode where calibration curves were generated as low as 25 parts per trillion. A similarly MIP-modified electrode was able to perform detection via impedance spectroscopy at about 1.7 parts per trillion, see, e.g., Clark, Rebecca, and Jeffrey Dick, “Electrochemical Sensing of Perfluorooctanesulfonate (PFOS) Using Ambient Oxygen in River Water”, ACS Sensors 5:3591-359, 2020. Similar low detection limits have been seen in other studies utilizing MIP-modified electrodes, see, e.g., Kaemi, Rezvan, Emili Potts, and Jeffrey Dick, “Quantifying Interferent Effects on Molecularly Imprinted Polymer Sensors for Per- and Polyfluoroalkyl Substances (PFAS)”, Analytical Chemistry 92:10597-10605, 2020; and Lu, Dingnan, David Zhu, Huihui Gan, Zhiyuan Yao, Jiayue Luo, Shiran Yu, Pradeep Kurup, “An ultra-sensitive molecularly imprinted polymer (MIP) and gold nanostars (AuNS) modified voltammetric sensor for facile detection of perfluorooctance sulfonate (PFOS) in drinking water”, Sensors and Actuators B: Chemical 352:1310, 2022. However, commercially available services to provide these types of electrodes are not currently available as of this writing and could prove to be problematic for the eventual fielding of a such a sensor.

Bubble nucleation evaluation has also been a reported electrochemical analysis with low levels of PFAS detection. In this method, the potential of the electrode is lowered until hydrogen gas is generated. The interaction of PFAS (as a surfactant) with the stabilization of the gas bubble is detectable as an electrical current shift during the bubble nucleation process. Methods utilizing this phenomenon have been reported to provide limits of detection down to 40 parts per trillion with a preconcentration step, see, e.g., Ranaweera, Ruchiranga, Carina Ghafari, and Long Luo, “Bubble-Nucleation-Based Method for the Selective and Sensitive Electrochemical Detection of Surfactants”, Analytical Chemistry 91:7744-774, 2019; and Ranaweera, Ruchiranga, Shizhong An, Yue Cao, and Long Luo, “Highly efficient preconcentration using anodically generated shrinking gas bubbles for per- and polyfluoroalkyl substances (PFAS) detection”, Analytical and Bioanalytical Chemistry, 2022. This bubble nucleation method requires nanoelectrodes, see, e.g., An, Shizhong, Ruchiranga Ranaweera, and Long Luo, “Harnessing bubble behaviors for developing new analytical strategies”, Analyst 145:7782-779, 2020, which may not be commercially available or robust enough for a field portable system.

Accordingly, it is important to be able to readily test wastewater in the field, whether or not further contaminated with oil or grease, for the presence or absence of PFAS for possible further treatment. In view of the foregoing and for other reasons that will become evident, there exists a need in the art to streamline the confirmation/testing process by developing an analytical platform that could be deployed in the field (e.g., OCONUS) to aid in confirming the presence or absence of PFAS in wastewater and also in OWW whether from a bilge or from other contaminated water sources.

### SUMMARY OF THE INVENTION

An embodiment of a system for detecting per- and polyfluorinated compounds (PFAS) in wastewater is disclosed. The system may include an optional sample collection container for storing a volume of the wastewater and from which a sample of the wastewater may be obtained, a salinity subsystem configured for measuring and adjusting salinity of the sample wastewater to obtain a proper salinity sample of the wastewater, an optional electrocoagulation subsystem configured for cleaning oil and grease contaminants, if necessary, from the proper salinity sample of the wastewater to obtain a clean proper salinity sample of the wastewater, and a PFAS detection subsystem configured for detecting concentration of the PFAS in the clean proper salinity sample of the wastewater and reporting same to a user.

An embodiment of a method for field testing wastewater to detect per- and polyfluorinated compounds (PFAS) is disclosed. The method embodiment may include providing a system for detecting the PFAS in wastewater, obtaining the wastewater from its source, collecting a sample of the wastewater for field analysis. The method embodiment may further include determining whether the sample has suitable salt content, if not, then adjusting salinity of the sample to a proper salinity content by adding salinity adjustment solution to the sample to obtain a proper salinity sample, if yes, then continuing with the proper salinity sample. The method embodiment may further include determining whether the proper salinity sample has oil or grease contamination; if yes, then applying an electrocoagulation treatment, settling and separating an oil contamination portion of the proper salinity sample, and continuing with a remaining portion of a clean proper salinity sample; if not, then continuing with the clean proper salinity sample. The method embodiment may further include extracting a small volume sample from the clean proper salinity sample, adding a pH adjustment and buffer solution to the clean proper salinity small volume sample to obtain a field sample ready for analysis, preparing a reference chemical solution to obtain a reference sample, establishing a baseline electrochemical activity on the reference sample, determining a field sample electrochemical activity on the field sample, and comparing the field sample electrochemical activity to the baseline electrochemical activity to determine if the field sample is contaminated with PFAS according to the field testing.

An embodiment of a field kit for detecting per- and polyfluorinated compounds (PFAS) in wastewater is disclosed. The field kit may include a storage and transportation container for storing and transporting the field kit, a sample collection container for storing a volume of the wastewater and from which a sample of the wastewater may be obtained, a salinity subsystem configured for measuring and adjusting salinity of the sample wastewater to obtain a proper salinity sample of the wastewater, an optional electrocoagulation subsystem configured for cleaning oil and/or grease from the proper salinity sample of the wastewater, if necessary, to obtain a clean proper salinity sample of the wastewater, and a PFAS detection subsystem configured for detecting presence and concentration of the PFAS in the clean proper salinity sample of the wastewater.

### BRIEF DESCRIPTION OF THE DRAWINGS

The following drawings illustrate exemplary embodiments for carrying out the invention. Like reference numerals refer to like parts in different views or embodiments of the present invention in the drawings.

FIG. 1 is diagram illustrating the process of electrocoagulation, according to the present invention.

FIG. 2 is a side view of an exemplary electrocoagulation electrode assembly, according to the present invention.

FIG. 3 is an image of an exemplary electrocoagulation subsystem, according to the present invention.

FIG. 4 is a high-level block diagram of a system for detecting PFAS, according to the present invention.

FIG. 5 is an exemplary graph illustrating baseline differential pulse voltammetry (DPV) activity measurements of a reference solution versus activity measurements of clean water, according to the present invention.

FIG. 6 is an exemplary graph illustrating baseline DPV activity measurements of a reference solution, in this instance an OWW sample without PFAS after cleaning by electrocoagulation versus DPV activity measurements of the OWW sample with PFAS, according to the present invention.

FIG. 7 is a diagram illustrating primary components included in a PFAS detection subsystem of a kit embodiment of the present invention.

FIG. 8 is a flowchart of an embodiment of a method for detecting PFAS in wastewater, according to the present invention.

### DETAILED DESCRIPTION

The disclosed methods and systems below may be described generally, as well as in terms of specific examples and/or specific embodiments. For instances where references are made to detailed examples and/or embodiments, it should be appreciated that any of the underlying principles described are not to be limited to a single embodiment but may be expanded for use with any of the other methods, apparatuses and systems described herein as will be understood by one of ordinary skill in the art unless specifically otherwise stated.

Given the toxicity of PFAS and the growing concern for it in our drinking water, oceans, and lakes, there is an urgent need for new methods that can detect PFAS at low cost, size, weight, power, and/or sample volume. As noted above, there is a present need to easily measure the concentrations of emerging contaminants, such as PFAS found in various wastewater sources including bilgewater as both international and domestic regulatory scrutiny increases for these compounds.

PFAS-impacted water disposal in foreign ports requires special handling that is consuming considerable operational resources, affecting mission readiness of naval assets in strategic locations. Currently, in countries which do not permit the discharge of OWW until PFAS concentrations are verified to be below regulatory values, OWW is containerized and sampled. Samples are then sent to certified commercial laboratories that can perform the specialized analyses needed to measure PFAS and related compounds. The complexity of the bilgewater matrix further complicates the analysis through introduction of potential interferences, resulting in possible data quality issues and long turn-around times for results. This process has resulted slowdown of OWW disposal in affected ports, causing logistical and operational challenges.

The inventors disclosed methods for determining the presence of perfluorooctanesulfonic acid (PFOS) in OWW in Hsu, Lewis, Richard Ordonez, Cody Hayashi, Nicholas Hayman, Mario Malfavon, Channing Bolt and Robert George, “Preliminary Considerations for Electrochemical PFOS Detection in Oily Wastewater”, Naval Information Warfare Center (NIWC) Pacific, Technical Report 3296 November 2022, the contents of which are hereby incorporated by reference for all purposes as if full set forth herein. While electrocoagulation techniques were found promising in that Technical Report, electrochemical detection of the PFOS was left as future work. The inventors further assessed the use of various techniques including electrocoagulation for removal of oil and/or grease in OWW in Hsu, Lewis, Richard Ordonez, Cody Hayashi, Mario Malfavon, Channing Bolt, Nicholas Hayman and Robert George, “Initial Determination of Electrocoagulation as an Oily Wastewater Treatment Technique at Shore Facilities”, OCEANS 2023—MTS/IEEE, 25-28 Sep. 2023, the contents of which are hereby incorporated by reference for all purposes as if full set forth herein.

The present invention solves the technical problem of detecting the presence of PFAS-impacted wastewater that may or may not also include oil and/or grease contamination. The present invention includes systems, methods and field portable kits for detecting per- and polyfluoroalkyl substances (PFAS), including PFOS and related chemicals, in wastewater and also oily wastewater (OWW) in the field with little to no laboratory space requirements.

Method embodiments of the present invention provide the ability to perform low-cost detection of analytes such as PFAS in a liquid medium with a disposable, portable, and rugged system. The method, system and kit embodiments only require a limited amount of oil and grease free wastewater solution (~microliters) to determine if an analyte is present or not. A particular embodiment of the present invention includes a field transportable kit for determining the presence or absence of PFAS contaminants. This field kit embodiment of the present invention includes advanced portable electrochemical instruments to monitor for the presence of PFAS at various concentration levels. The present invention provides the ability to pre-treat a sample of OWW in the field prior to preparing the sample for electrochemical measurement. The method also accounts for various salinities and provides options to use non-hazardous, readily available chemicals for detection.

The inventors' solution to the technical problem of detecting the presence of PFAS in oily wastewater begins with separating the oil and grease, if present, from the OWW. The inventors employ electrocoagulation techniques to separate out the oil and/or grease from OWW. Once “clean” wastewater is obtained, the present invention proceeds by determining the presence or absence of PFAS contaminants in the remaining “clean” wastewater. Further detailed description of the inventive techniques, systems and kits for detecting the presence of PFAS in wastewater follows.

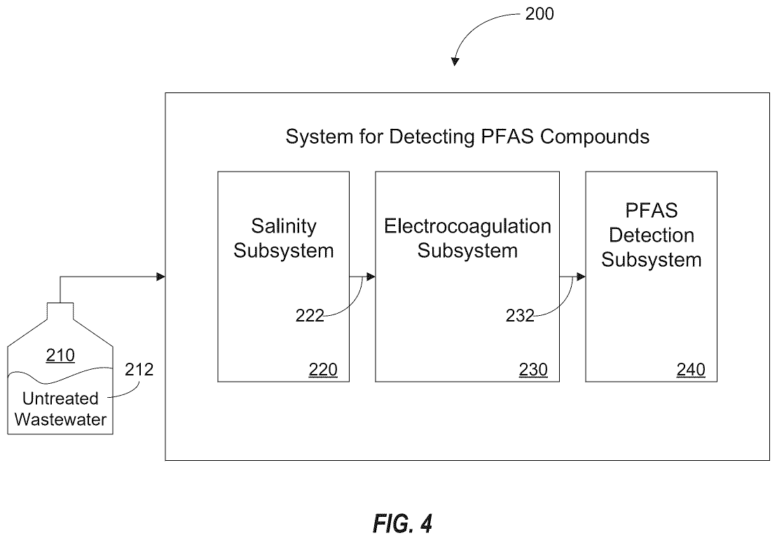

FIG. 4 is a high-level block diagram of a system 200 for detecting PFAS in wastewater, according to the present invention. As shown in FIG. 4, an optional liquid storage container 210 may be included in system 200 for storing untreated wastewater 212, which may be OWW. System 200 may further include a salinity subsystem 220 configured for measuring and adjusting the salinity of the untreated wastewater 212 the output of which is a proper salinity wastewater sample 222. System 200 may further include an electrocoagulation subsystem 230 which takes the proper salinity wastewater 222 and removes the oil and/or grease if the proper salinity wastewater 222 is OWW. The output of the electrocoagulation subsystem 230 is a clean proper salinity sample 232. With a clean proper salinity sample 232, the system for detecting PFAS 200 is ready to perform the final step of detecting the presence and concentration of PFAS. Accordingly, system 200 may further include a PFAS detection subsystem 240. Each of these subsystems are described in further detail, below.

### Salinity Measurement and Adjustment

As shown in FIG. 4, the salinity subsystem 220 of the present invention is important for measuring and adjusting the salinity of the initial untreated wastewater, 212, especially where the untreated wastewater 212 is OWW. There are a number of methods that may be employed for measuring and adjusting the salinity of OWW to determine if there is sufficient salinity (ionic strength) for efficiently performing the electrocoagulation process. Essentially, anything that allows for an electrical resistance measurement of the OWW solution may be used, e.g., a beaker or other container with a known volume of OWW solution, electrodes with set geometry and spacing, such as the electrocoagulation electrode assembly 60 disclosed herein and a digital multimeter, see, e.g., Klein Tools, Digital Multimeter, Model No. MM325, available from various distributors. Another optional instrument useful for performing a salinity measurement may include a salinity meter, see, e.g., Hach Company, Model 9532700, Pocket Pro+Multi 1 Tester for Conductivity/Salinity with replaceable sensor, available from various vendors including Amazon. Alternative conductivity/salinity measurement devices such as those operating on optical principles (refractometer) or by liquid specific gravity may also be used consistent with the teachings of the present invention and are available from similar vendors.

For the oil/grease treatment by electrocoagulation and for the actual PFAS detection method by electrochemistry, it is desirable to minimize any “solution resistance” to charge transfer. This may be accomplished by adding enough ions to the solution so that charge transfer is “easy” and solution resistance does not impact electrochemical processes. Adjustment of the OWW solution salinity may be performed according to any suitable method. While there are a number of known techniques for adjusting salinity, two of the most convenient are simply adding potassium phosphate or potassium chloride as a salt addition. However, it will be understood that any easily dissolvable salt may be used as long as it does not interact with other species in solution. For example, and not by way of limitation, packets of salt (NaCl or KCl, e.g., potassium chloride 20 mEq oral packet, available from Kaiser Permanente, model 0245-0360-30) may be added to the solution to increase salinity. Alternatively, selected amounts of deionized water may be added to the solution to decrease salinity. Generally, salinity adjustment is primarily to add salinity. A salinity of about 0.1M of salt is presently preferred. However, other ranges of salinity will also work and fall within the scope of the present invention. The particular details of performing such a salinity measurement and making a suitable adjustment for proper salinity will be within the knowledge of one of ordinary skill in the art and thus will not be further elaborated herein. As noted above, this optional salinity measurement and adjustment process step is preferably performed prior to the electrocoagulation step, if necessary. However, salinity adjustment can also be performed after electrocoagulation according to other embodiments.

### Emulsion Destabilization and Physiochemical Properties of OWW

The process of emulsion destabilization is affected by various physiochemical properties of the OWW, see, e.g., Church, et al., supra. The inventors found four physiochemical properties of the OWW found in bilgewater to be particularly relevant. First, in the simplest of emulsions, e.g., deionized (DI) water and oil, increased temperature results increased oil droplet movement and collision, generally resulting in destabilization. However, in OWW, which has all sorts of other contaminants including surfactants, this increase in molecular collision due to increased temperature may increase the effectiveness of the surfactants in stabilizing the emulsification, see, e.g., Kokal, Sunil, “Crude-Oil Emulsions: A State-Of-The-Art Review”, SPE Production & Facilities 5-13, 2005.

Second, the types of surfactants and their concentration can also decrease the rate of emulsion destabilization, depending on the specific surfactant and concentration. Destabilization can be complicated by the fact that bilgewater surfactant loads are not well characterized and are highly variable depending on daily shipboard activities, such as cleaning or even fire suppression.

Third, pH can influence the stability of these solutions in complex ways, typically with higher pH resulting in greater destabilization, with typical bilgewater pH reported between 6.8 to 9.0, and likely to frequently change due to chemical composition of the bilgewater. For this reason, a pH adjustment and buffer solution step is presently preferred in the embodiments (system, method and field kit) of the present invention where the wastewater sample falls outside a preferred pH range. The primary goal for making a pH adjustment is to ensure the PFAS can be completely dissociated to its negative ion state. The negative ion state is important to the method of detection because a step in the procedure is to adsorb PFAS to the electrode by creating a positive charge on the electrode surface with a potentiostat before applying a differential pulse voltammetry (DPV) measurement. When the PFAS ion attaches to the electrode it decreases the available surface to perform electrochemistry. This decrease is proportional to the concentration of PFAS and thus indicates the presence of PFAS as well as concentration. Accordingly, in order to measure the decrease, the method employs taking a baseline measurement first with the reference solution before measuring the sample.

For PFAS compounds that can be completely dissociated, generally at neutral pHs the proton is not associated with the main compound and the ion maintains a negative charge. The acid dissociation constant of most PFAS is below 4. Accordingly, the target is a neutral pH, but any pH above 6 will also suffice according to the present invention. Any suitable buffer solution may be used according to the present invention. A presently preferred buffer solution is a phosphate buffer since a phosphate buffer is fairly easy to make and use at higher salt concentrations. In a particularly useful embodiment of the present invention, a phosphate buffer may be used as both a salinity adjustment and as a pH buffer.

Finally, ionic strength can be especially important in the stability of emulsions. Specifically, increasing ionic strength can shield electrostatic repulsion from the oil droplets, resulting in coalescence and flocculation. In addition, salts can impact surfactant solubility and can increase the density of the aqueous phase. An easy measurement of ionic strength is electrical conductivity and conductivities of bilgewater have been reported in the range of 679 μS/cm to 8.4 mS/cm, see, e.g., Church, et al., supra. Accordingly, presently preferred embodiments (system, method and field kit) of the present invention may include an optional salinity measurement and adjustment prior to removal of the oil and grease that may be found in OWW. It will be understood that bilge water or other OWW may inherently have sufficient salinity to move forward with efficient electrocoagulation.

### Electrocoagulation

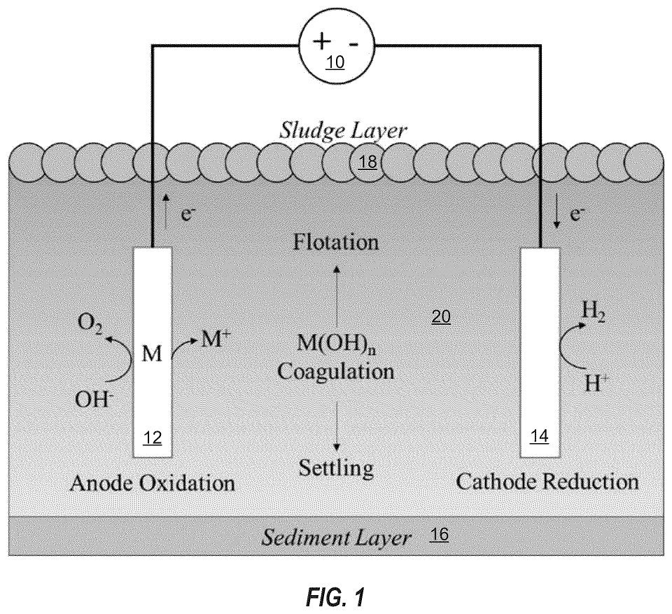

In the present invention, electrocoagulation is used to neutralize repulsive electrostatic forces, allowing for flocculation via attractive van der Waals forces. The electrocoagulation subsystem 230 shown in FIG. 4 is used to remove oil and/or grease from proper salinity wastewater 222. Electrocoagulation, the process of applying an electrical current through electrodes to separate contaminants in a liquid, can be used to separate oil and grease from OWW, see, e.g., Rincon, Guillermo J, and Enrique J La Motta, “Simultaneous removal of oil and grease, and heavy metals from artificial bilge water using electro-coagulation/flotation”, Journal of Environmental Management 144:42-50, 2014. FIG. 1 is diagram illustrating the process of electrocoagulation, according to the present invention. As shown in FIG. 1, metal anode electrode 12 on the left is oxidized to form metal hydroxide, M (OH) n, flocs that are removed via flotation or settling. More particularly, FIG. 1 illustrates a power supply 10 connected to electrolysis electrodes, namely an anode 12 and cathode 14 submerged in an oily wastewater sample 20. Upon an application of voltage/current, the oil and/or grease contaminants may settle in a sediment layer 16 or float up to a sludge layer 18.

Oxygen (O₂) and hydrogen (H₂) may also be formed at the electrode surfaces due to hydrolysis. The electrocoagulation process entails oil/grease contaminant removal similar to coagulation-flocculation process except that no additional chemicals are required. Metal ions are provided by electrochemical oxidation of an iron or aluminum electrode through an applied voltage or current. An exemplary electrocoagulation process may include the following steps: (1) An electrical current may be applied to electrolysis electrodes 12, 14 via a power supply 10. (2) The electrolysis electrodes generate metal ion coagulants due to oxidation of the electrode metal. (3) Contaminants destabilize, particulates are suspended, and emulsions break during the application of the electrical current. (4) Flocs (aggregation of destabilized particles) form suspended in the solution. (5) Hydrolysis (separation of the oxygen and hydrogen in the water) occurs and bubbles emerge to lift flocs to the surface of the liquid. (6) Smaller bubble sizes allow more effective separation of contaminants from water that rise up to the surface as a foamy sludge layer 18. (7) Another fraction of the contaminants settle to the bottom in a sediment layer 16. The “clean” wastewater remains after the foamy sludge 18 and sediment 16 layers are removed from the original wastewater solution, leaving clean wastewater.

Several variables can affect the electrocoagulation process and can be preselected depending on the targeted liquid medium. On such variable is the material used in the electrocoagulation electrode. Such electrodes typically consist of carbon, steel, aluminum, and/or iron electrodes due to their cost effectiveness. However, other metals such as platinum/iridium, titanium, and stainless steel can also be utilized for the electrode material. The magnitude of the electrical current is another electrocoagulation process variable. The magnitude of electrical current flow between the electrodes is directly related to the amount of floc produced and the ability to separate contaminants from the solution. The electrical current can be altered through changing the applied voltage, electrode geometry, electrode separation distance, and/or solution electroconductivity. The flow rate of the wastewater liquid is yet another variable. Flow rate of the liquid can be controlled with a stirrer, or similar, and improve the amount of contaminant separation during electrocoagulation, see, e.g., El-Shazly, Ahmed, and Mohammad Daous, “Investigations and Kinetics Study for the Effet [sic] of Solution Flow Rate on the Performance of Electrocoagulation Unit Used for Nutrients Removal”, International Journal of Electrochemical Science, 8:12509-12518, 2013.

Because of the significant number of variables associated with the electrocoagulation process, the inventors analyzed several approaches similar to their desired use case—particularly examples that performed electrocoagulation on marine oily wastewater, see, e.g., Han, Meiling, Jin Zhang, Wen Chu, Jiahao Chen, and Gongfu Zhou, “Research Progress and Prospects of Marine Oily Wastewater Treatment: A Review”, Water 11 (12):2517, 2019; Church, Jared, Jeffrey Lundin, Daniela Diaz, Dianne Mercado, Marjorie Wilner, Woo Hyoung Lee, and Danielle Paynter, “Identification and characterization of bilgewater emulsions”, Science of The Total Environment 691:981-995, 2019; Waller, Gordon, Curtis Martin, Nicholas Jones, and Danielle Paynter, “Treatment of oil-in-saltwater emulsions by in-situ production of magnetic FeOx nanoparticles”, Journal of Water Process Engineering 31:100851, 2019.

Table 1, below, summarizes some of the published reports utilizing aluminum as the electrode material for oxidation in an electrocoagulation process.

TABLE 1
Pollutant removal efficiencies of OWWs via aluminum electrode electrocoagulation

| Electrochemical | | | |
|---|---|---|---|
| Treatment | Oily | | |
| Method | wastewater | Result | Reference |
| Aluminum | Real | 81.74% Oil- | Ulucan, Kubra, Harun Akif Kabuk, Fatih |
| | bilgewater | grease | Ilhan, and Ugur Kurt, “Electrocoagulation |
| | | Removal | Process Application in Bilge Water |
| | | | Treatment Using Response Surface |
| | | | Methodology”, International Journal of |
| | | | Electrochemical Science 9: 2316-2326, |
| | | | 2014. |
| Steel/Aluminum | Synthetic | 99.9% Oil- | Rincon, Guillermo J, and Enrique J La |
| | | grease | Motta, “Simultaneous removal of oil and |
| | | Removal | grease, and heavy metals from artificial |
| | | | bilge water using electro- |
| | | | coagulation/flotation”, Journal of |
| | | | Environmental Management 144: 42-50, |
| | | | 2014. |
| Aluminum | Real | 81% Oil- | Sekman, Elif, Gamze Varank, Selin Top, |
| | wastewater | grease | Mehmet Sinan Bilgili, and E Uslu, |
| | | Removal | “Treatment of Oily Wastewater From Port |
| | | | Waste Reception Facilities by |
| | | | Electrocoagulation”, International Journal of |
| | | | Environmental Research 5 (4): 1079-1086, |
| | | | 2011. |
| Aluminum | Synthetic | 98.8% | Safari, Sania, Mojtaba Azadi Aghdam, and |
| | wastewater | diesel | Hamid-Reza Kariminia, “Electrocoagulation |
| | | removal | for COD and diesel removal from oily |
| | | | wastewater”, International journal of |
| | | | Environmental Science and Technology 13: |
| | | | 231-242, 2015. |
| Aluminum | Real | 83.91% | AlJaberi, Forat Yasir, “Removal of TOC |
| | wastewater | Total | from oily wastewater by electrocoagulation |
| | | Organic | technology”, IOP Conf. Series: Materials |
| | | Carbon | Science and Engineering 928, 2020. |
| | | removal | |
| Aluminum | Synthetic | 94% oil- | Al-Rubaiey, Najem, and Mohammed Al- |
| | wastewater | grease | Barazanjy, “Electrocoagulation Treatment |
| | | removal | of Oily Wastewater in the Oil Industry”, |
| | | (as | Journal of Petroleum Research and Studies |
| | | turbidity) | 8 (3): 274-289, 2018. |

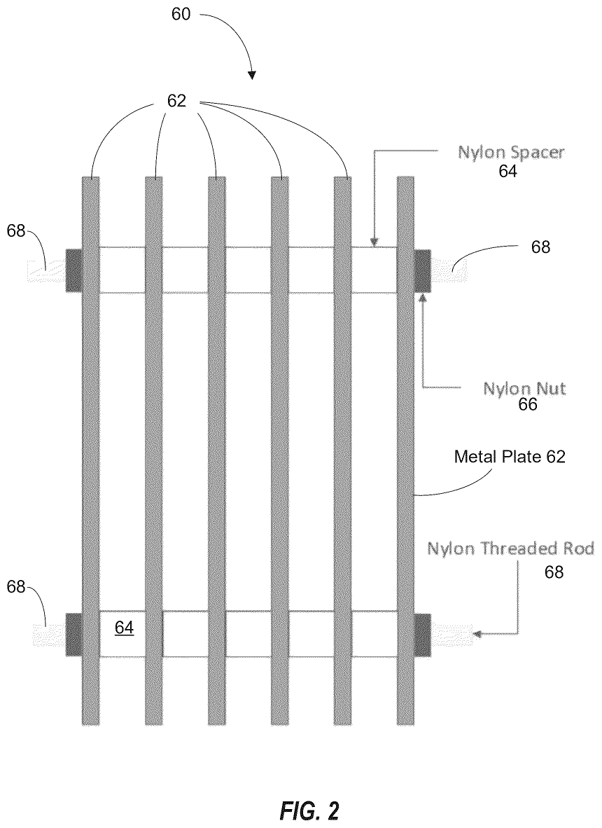

For the illustrated embodiments of the present invention, aluminum is a presently preferred electrocoagulation electrode metal used in the electrocoagulation subsystem 230 shown in FIG. 4. However, it will be understood that any suitable electrocoagulation electrode material, e.g., carbon, steel, platinum/iridium, titanium, and stainless steel and iron electrodes, may also be used consistent with the teachings of the present invention. FIG. 2 is a side view of an exemplary electrocoagulation electrode assembly 60, according to the present invention. The exemplary electrocoagulation electrode assembly 60, may include a plurality of metal bars 62 separated by insulating spacers 64 and assembled using threaded insulating support rods 68 and insulating nuts 66. Though six metal bars 62 are illustrated in FIG. 2, it will be understood that any suitable number of metal bars 62 may be employed consistent with the teachings of the present invention. According to a presently preferred embodiment, the metal bars 62 may be formed of aluminum and the threaded insulating rods 68, spacers 64 and nuts 66 may be formed of a plastic material, e.g., nylon. The multiple metal bars 62 alternate between anode and cathode according to connection to a power supply (not shown) in order to provide greater surface area for the electrocoagulation process.

An exemplary electrocoagulation electrode assembly 60 may be constructed from 0.3175 cm (⅛ inch) thick aluminum flat bar stock. According to one embodiment, the aluminum was machined to form 6.35 cm (2.5 inch) wide×15.24 cm (6 inch) long plates to form the metal bars 62. A 0.635 cm (¼ inch) hole was drilled 1″ from the bottom and top edges and at the center of each metal bar 62 in order to fit threaded insulating support rods 68 to hold the metal bars 62 in place. A total of 6 metal bars 62 were used for the exemplary electrocoagulation electrode assembly 60. However, it will be understood that various quantities of metal bars having various dimensions may also be used to form the metal bars 62 used in an electrocoagulation electrode assembly 60, according to other embodiments of the present invention. At a minimum a positive and a negative electrode would be required. But, other arrangements that included unpaired electrodes, or electrodes that were not interdigitated are also withing the scope of the present invention. According to a particular embodiment, ¼-inch threaded nylon rod was used to form the threaded insulating support rods 68 along with ½-inch nylon spacers forming the insulating spacers 64 to ensure the distance between the metal bars 62 would remain consistent. The insulating nuts 66 were fitted on the ends of each threaded insulating support rod 68 to complete the exemplary electrocoagulation electrode assembly 60.

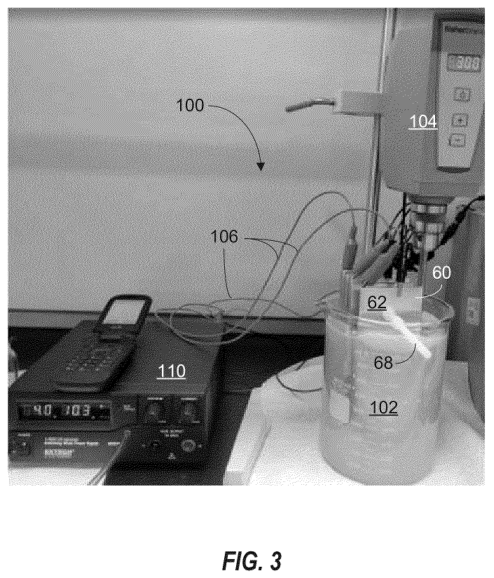

FIG. 3 is an image of an exemplary electrocoagulation subsystem 100, according to the present invention. FIG. 3 illustrates an electrocoagulation electrode assembly 60 including 6 metal bars 62 placed in a glass beaker 102 containing OWW and suspended so that the metal plates 62 are only partially submerged. Leads 106 with stainless steel alligator clips are shown connected to the top of the metal plates 62 to provide electrical connection to a laboratory power supply 110. Any suitable connecting leads 106 may be used, see, e.g., Eisco Labs PK12 Connecting Leads, Product No. PH1053APK12, which come in red or black to track anode and cathode leads, see also, Pomona® Model 1166 & 6357 Kit Alligator Clip to Multi-Stacking Banana Plug Patch Cord, available from various distributors, e.g., Digi-Key Corp., Mouser Electronics, etc. However, it will be understood that any suitable means of making an electrical connection between the power supply 110 and the electrocoagulation electrode assembly 60 are contemplated to be within the scope of the present invention. The metal plates 62 are not entirely submerged to ensure that the alligator clips attached to one end of the leads 106 do not react in the OWW during voltage application. An extended length (exceeding beaker diameter) of the upper threaded insulating rod 68 may be used to suspend the electrocoagulation electrode assembly 60 within the beaker 102 of OWW. A power supply 110 delivers power to the plurality of metal bars 62 in the electrocoagulation electrode assembly 60. The embodiment of power supply 110 illustrated in FIG. 3 is an Extech Instruments® Model 382275, available from Teledyne Flir, LLC, Wilsonville, OR. However, it will be understood that any suitable power supply 110 may be used consistent with the teachings of the present invention, see e.g., Keithley Model No. 2200-60-2, Programmable DC Power Supply, available from TEquipment, 205 Westwood Avenue, Long Branch, NJ 07740. Polarity of the metal plates 62 is alternated (interdigitated) such that a negatively charged plate is always adjacent to a positively charged plate, according to the embodiment illustrated in FIG. 3. The use of a stirrer or an agitator 104 to circulate the OWW, though optional, is also preferred to enhance the chemical reactions.

### PFAS Detection

As illustrated in FIG. 4, the PFAS detection subsystem 240 analyzes the clean proper salinity sample 232 for PFAS detection and concentration. In view of the shortcomings associated with other conventional techniques for detecting PFAS as described herein, the inventive system, method and kit of the present invention utilizes screen-printed electrodes (SPEs) for PFAS compound detection. However, it will be understood that any suitable configuration of electrochemical detection electrodes consistent with the teachings of the present invention are perceived to be with the scope of the present invention. The singular term “screen-printed electrode” (SPE) as used herein refers to an integrated assembly of standard electrochemical cell electrodes, i.e., working, counter, and reference, placed onto a small-footprint substrate, as described in more detail below.

A presently preferred screen-printed electrode (SPE) used in the system, method and kit embodiments of the present invention is sourced from Metrohm® DropSens and is an electrochemical sensing platform. Each Metrohm® DropSens SPE includes the standard electrochemical cell electrodes, i.e., working, counter, and reference, placed onto a small-footprint substrate. Each Metrohm® DropSens SPE further includes a connector to the three cell electrodes and a 200 μL capacity well for receiving the solution to be analyzed. Various metal and other conductive material inks may be used to form the electrodes of each SPE and can be customized for particular analytes of interest, for example, silver, carbon, gold, platinum, ruthenium oxide, aluminum, cobalt, chromium, indium tin oxide, molybdenum, lead, palladium and tungsten. While there are a wide variety of available electrode materials that can be used to form the electrodes of the present invention, platinum, palladium and silver are the presently preferred SPE electrode metals for the detection of PFAS given their stronger adsorption. Note that the method of the present invention relies on the PFAS compound being absorbed to the metal electrodes and changing the electrical properties of the electrodes. Accordingly, platinum, palladium and silver appear to provide the greatest change in electrical properties in response to PFAS. It will be further understood that the surface roughness, chemical properties or the electrochemical sensor electrodes may be selectively modified to increase PFAS adsorption and thus increase sensitivity to PFAS in solution, according to other embodiments of the present invention. Similarly, altering the geometry of the electrochemical cell electrodes, e.g., to increase surface area or shape, may also be used to achieve greater PFAS sensitivity according to additional embodiments of the present invention.

The Metrohm® DropSens SPEs are each configured with a connector, opposite the well, for plugging into a port of an electrochemical reader, namely, a Metrohm® DropStat. The Metrohm® DropStat is a hand-held, single-technique, potentiostat-based custom electrochemical reader that is configured to perform preselected voltammetry measurements and display the concentration of the particular analyte for which an electrochemical sensor has been specifically developed. According to the method of detecting PFAS in a “clean” wastewater sample (i.e., uncontaminated, no oil/grease) of the present invention, the process begins with establishing a reference baseline electrochemical response with a new SPE, followed by rinsing the SPE well with DI, and then apply the sample solution to by analyzed to the rinsed well and performing a second electrochemical response. The use of a single SPE is intentional in the inventive methodology. By using a single SPE for both measurements, any variability between electrodes that might occur is eliminated, since SPEs may have some degree of variability from electrode to electrode. The following is a particular method of performing the PFAS compound detection. According to other embodiments, SPEs may be Metrohm® DropSens SPEs or custom made and used with other potentiostats to perform the DPV measurements described herein, e.g., Gamry Instruments, 734 Louis Drive, Warminster, PA 18974, and others known to those of ordinary skill in the art.

Given the 200 μL capacity well, a working volume 150 μL may be targeted for delivery to the SPE well in order to avoid overflowing. For the reference and sample solution preparation and electrochemical measurements the following is an exemplary procedure:
1. Aliquot 180 μL of reference in a 1.5 mL centrifuge tube & 180 μL of the sample in another 1.5 mL centrifuge tube.
2. Add 10 μL of pH/salinity adjustment (20×) solution; 10 μL reporter chemical (20×) solution.
3. Vortex 10 min to mix adjustment and reference solution (reporter) chemicals in both solutions.
4. Condition screen-printed electrode (SPE) with blank buffer solution.
5. Perform electrochemical measurement on reference solution (150 μL) with buffer prepared SPE.
6. Remove any remaining reference liquid via wicking, e.g., by Kimwipe.
7. Add ultrapure (DI) water and remove by wicking (3 times) to rinse SPE well.
8. Perform electrochemical measurement on sample (150 μL).
9. Discard SPE.

The reference solution (sometimes referred to as a “reporter chemical”) is any chemical with a well-known electrochemical behavior. For example, and not by way of limitation, a solution of copper sulfate, or potassium ferricyanide solution may be used as a reference solution. Potassium ferricyanide solution is the presently preferred reference solution for the method, system and kit embodiments for detecting PFAS of the present invention. Potassium ferricyanide electrochemistry is well understood, reversible, and does not decompose within the voltage window used in the present invention. The addition of the reference solution (reporter chemical) provides an electrochemical signal during the measurements of both the reference solution and the sample. PFAS are generally inert, so they generally would not provide a signal during an electrochemical test. The pH/salinity adjustment and reference solution are added to both centrifuge tubes prior to vortex (step 2, above).

The electrochemical measurements noted above are differential pulse voltammetry (DPV) measurements performed by a Gamry Interface 1010E potentiostat, or alternatively with a the Metrohm® DropStat, with results downloaded to a processor (laptop) via a cable for post-processing of the measurements for PFAS detection and concentration determinations and graphical display to a user. It will be understood that the above is merely a particular procedure according to the present invention and that various adjustments and variations to the above may also be employed to detect PFAS according to the present invention.

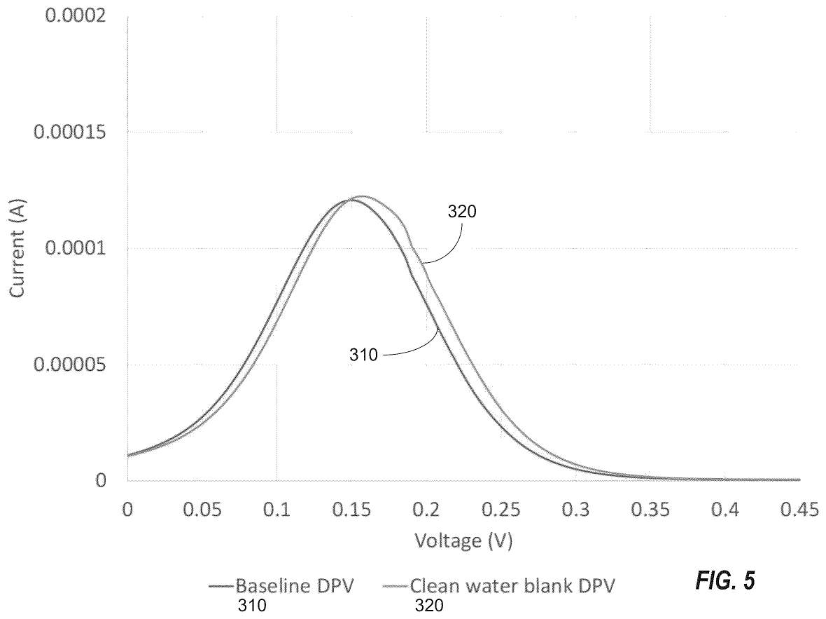

FIG. 5 is an exemplary graph illustrating baseline differential pulse voltammetry (DPV) activity measurements of a reference solution 310 versus baseline activity measurements of clean water (no PFAS, or blank) 320, according to the present invention. Both curves 310 and 320 are averages of replicate DPV experiments for both the reference 310 and clean water 320 measurements. FIG. 5 represents a blank sample for quality assurance/control purposes and forms a baseline of the system and method.

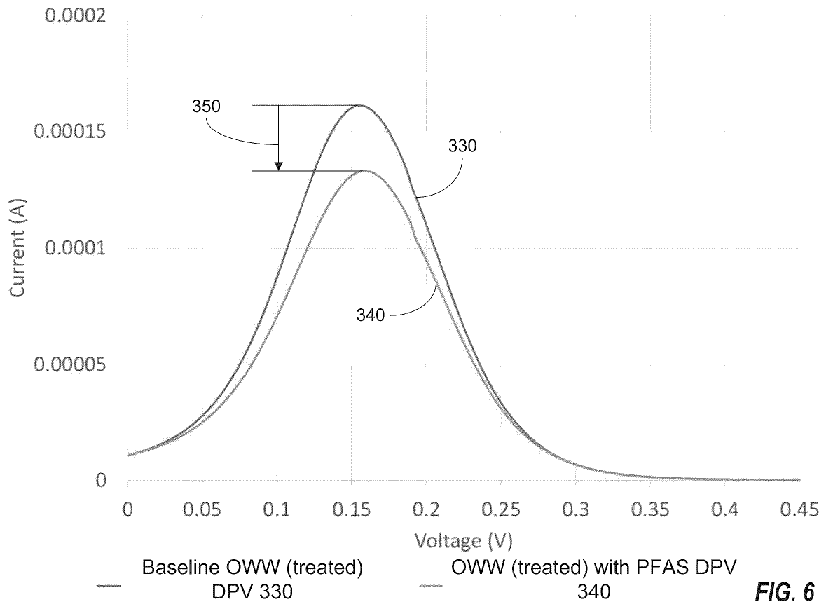

FIG. 6 is an exemplary graph illustrating baseline DPV activity measurements of a reference solution, in this instance an OWW sample without PFAS after cleaning by electrocoagulation 330 versus DPV activity measurements of the OWW sample with PFAS 340, according to the present invention. Again, both curves 330 and 340 are averages of replicate DPV experiments for both the reference OWW 330 (treated, or “oil removed”) and the OWW with PFAS 340 measurements. The observed decrease in activity shown in FIG. 6, see arrow 350, is a result of PFAS presence in the sample and the magnitude of the decrease can be correlated to the concentration of PFAS in the sample.

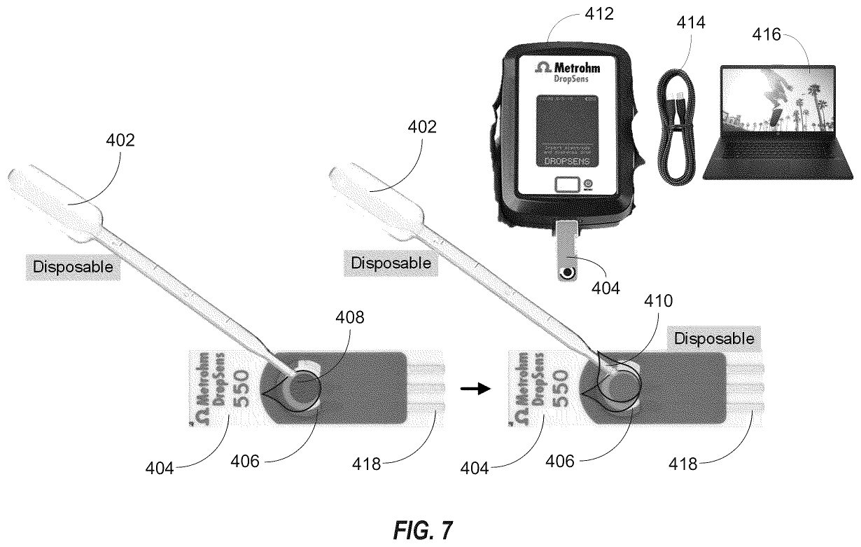

FIG. 7 is a diagram illustrating images of primary components included in a PFAS detection subsystem 340 (see FIG. 4) of a kit embodiment of the present invention. More particularly, FIG. 4 illustrates a couple disposable pipettes 402, one for depositing a reference solution 408 and the other for depositing the sample solution 410. FIG. 4 further illustrates a screen-printed electrode (SPE) 404 with a well 406 for receiving the reference 408 and sample 410 fluids delivered from the pipettes 402 and measure sequentially. The SPE 404 is used twice (indicated by arrow) before being discarded. FIG. 4 further illustrates an electrochemical reader 412 for performing potentiostat and DPV measurement techniques with the SPE 404 inserted. FIG. 4 further illustrates data transfer cable 414 for interfacing a processor 416 (e.g., a laptop computer). The SPE 404 shown in FIG. 7 illustrates the SPE connector end 418 of a Metrohm® DropSens SPE 404 that is shown inserted into electrochemical reader 412. It will be understood that the components illustrated in FIG. 7 are not drawn to scale.

Additional embodiments of kits for detecting PFAS compounds may be found in Tables 2 and 3 below. It will be understood that the equipment listed in Tables 2 and 3, may form components of either system or kit embodiments of the present invention and may be used in the method embodiments of the present invention.

More particularly, Table 2 provides useful components relating to the optional pre-processing phase, namely, components found in an electrocoagulation subsystem 230 (FIG. 4). Still more particularly, Table 2 summarized exemplary system and kit equipment used when the original wastewater appears to have a sheen or is otherwise obviously contaminated with oil, grease, sludge or other contaminant that is not of interest for testing of a given PFAS compound and that must be removed prior to PFAS compound detection.

TABLE 2
Kit components for an embodiment of an optional electrocoagulation subsystem

| Equipment | Purpose or Function |
|---|---|
| Liquid Storage Container | An optional vessel for storing enough of the |
| (optional) | wastewater sourced from bilge, or other |
| Any suitable sealable bottle, e.g., | wastewater source to perform test(s) for |
| Nalgene, glass, etc. | target contaminants (e.g., PFAS) |
| Beaker | Electrocoagulation vessel used to contain a |
| Any suitable glass beaker of suitable | volume of the wastewater sourced directly |
| size/volume to contain volume of | from bilge, or other wastewater source, or |
| wastewater and support an | from liquid storage container (above) and |
| electrocoagulation cell, and formed of | configured to receive the electrocoagulation |
| borosilicate glass, e.g., Brand ® glass | electrode assembly. |
| beaker with spout, low form, model | |
| BR91236; Pyrex Griffin; etc. | |
| Electrocoagulation Electrode | Performs the electrocoagulation of oily |
| Assembly | wastewater to separate oil/grease from the |
| Custom made as described herein, or any | original wastewater via voltage applied to |
| suitable equivalent. Electrode plates may | one or more cathode/anode pairs while at |
| be aluminum, or any other suitable | least partially submerged within the beaker |
| material. | with the original wastewater |
| Power Supply | Portable source of electricity for applying a |
| Keithly 2200 DC Power Supply, available | programmable voltage/current to the |
| from Tektronix, 13725 SW Karl Braun | electrocoagulation cell. |
| Drive, P.O. Box 500, Beaverton, OR; see | |
| also, Keithley Model No. 2200-60-2, | |
| Programmable DC Power Supply, | |
| available from TEquipment, 205 | |
| Westwood Avenue, Long Branch, NJ | |
| 07740 | |
| Leads | Wires/cables for delivering electricity |
| Eisco Labs PK12 Connecting Leads, | (voltage/current) from the power supply to |
| Product No. PH1053APK12, which come | the electrocoagulation electrode assembly, |
| in red or black to track anode and | which may also include alligator clips for |
| cathode leads; see also, Pomona ® Model | attachment to the electrocoagulation |
| 1166 & 6357 Kit Alligator Clip to Multi- | electrode assembly. |
| Stacking Banana Plug Patch Cord, | |
| available from various distributors, e.g., | |
| Digi-Key Corp., Mouser Electronics, etc. | |

It will be understood that Table 2, above, represents a nonexhaustive list of system or kit components for an optional electrocoagulation subsystem 230 (FIG. 4). Whereas, Table 3, below, is directed to system or kit components for a PFAS detection subsystem 330 (FIG. 4). More particularly, Table 3, below, summarized exemplary embodiments of equipment used in system and kit embodiments used to detect PFAS in “clean” wastewater, i.e., wastewater that is relatively free of oil and/or grease contaminants that if originally present are removed via the electrocoagulation processing step outlined above.

TABLE 3
Kit components for an embodiment of an PFAS detection subsystem

| Equipment | Purpose or Function |
|---|---|
| Pipettes (2, Disposable) | Fluid transfer devices: One for extracting a |
| Any suitable pipette, suction blub, or fluid | small volume (100-200 μL) of the |
| extraction device, etc., that may be used | reference solution for depositing on the |
| to extract 100-200 μL of the fluids to be | detection test electrode and measuring the |
| measured. An example transfer pipette | baseline, the other pipette for extracting the |
| suitable for use with the present invention | heterogenous “clean” wastewater, free from |
| is the 5 ML Uline, model S-24319 which | oil or grease sludge or sediment as a result |
| includes a squeezable and flexible LDPE | of the electrocoagulation process (above if |
| plastic body with bulb, available from | needed), for delivery to the screen-printed |
| Uline, 12575 Uline Drive, Pleasant | electrode (below), mixing and performing |
| Prairie, WI 53158. | the second voltammetry measurement for |
| | comparing to the first. Note that the clean |
| | wastewater will be found between the |
| | sediment layer at the bottom of the beaker |
| | and the foamy sludge layer floating at the |
| | top of the beaker from the |
| | electrocoagulation pre-processing phase |
| | (see, e.g., FIG. 1). |
| Screen-Printed Electrode (Disposable) | This electrode is configured with a window |
| Any suitable measurement electrode may | for depositing the fluid sample and a |
| be used. However, the presently | connector with all three cell electrodes for |
| preferred screen-printed electrodes are | insertion into an electrochemical reader |
| those sourced from Metrohm ® DropSens, | (below), which performs the voltammetry |
| e.g., Model 550 platinum electrode, or | analysis in conjunction with a processor |
| custom ordered with selected electrode | (below) with data transfer cable (below). |
| materials for absorbing PFAS as | |
| disclosed herein. | |
| Electrochemical Reader | The electrochemical reader performs the |
| The presently preferred electrochemical | voltammetry analysis on the sample |
| reader is the Gamry Interface 1010E | deposited on a screen-printed electrode |
| potentiostat. Alternative embodiments | (above) which has been inserted into the |
| may employ a Metrohm ® DropStat | reader. The reader provides data from the |
| portable electrochemical reader, which is | voltammetry measurement for post- |
| configured to use the Model 550 | processing by a processor (below). |
| electrodes (above) as well as any other | |
| Metrohm ® DropSens electrode and can | |
| deliver voltammetry data for post- | |
| processing via a USB-C port and cable. | |
| Processor | The processor (computer) is used for post- |
| Any suitable computer may be used | processing analysis of the voltammetry |
| consistent with the present invention. A | measurements performed by the |
| presently preferred embodiment is any | electrochemical reader and preferably |
| laptop computer with a USB-C port for | interfaces directly with the electrochemical |
| direct connection to the Gamry Interface | reader to download data and perform |
| 1010E potentiostat, or Metrohm DropStat | graphical and computational comparisons |
| electrochemical reader. An example | of the baseline measurements to the target |
| laptop computer is the HP Model 17t- | PFAS of interest and indirectly provide |
| cn300, available from Hewlett-Packard. | concentration estimates of same. The |
| Laptop portability is preferred for a | processor may also be configured with |
| transportable kit. | integrated software for performing the data |
| | transfer and analysis. |
| Data Cable | The data cable is used to transfer |
| The presently preferred data interface | voltammetry measurement data from the |
| cable for use between the | electrochemical reader to the processor |
| electrochemical reader and the processor | directly. Of course, wireless data transfer |
| is a USB-C to USB-C cable. An example | may also be used where the |
| is the Insignia ™ 6′ USB-C to USB-C | electrochemical reader is configured for |
| charge-and-sync cable, Model NS- | such support. |
| MCC621C, available from Best Buy. | |
| Reference Solution | The reference solution is a known chemical |
| Any suitable reference solution may be | (also referred to as a reporter chemical) |
| used consistent with the teachings of the | used to establish a baseline from which the |
| present invention. A presently preferred | sample is measured to determine PFAS |
| reference solution is potassium | presence and concentration. |
| ferricyanide. Alternative embodiments of | |
| reference solution may include copper | |
| sulfate. | |

The equipment described herein and shown in in Tables 2 and 3, above, assume access to alternating current (AC) power to run the equipment at a remote field location. An optional kit component particularly useful for field deployment is a storage container(s) for safely transporting and storing all of the equipment used to detect PFAS as described herein. It will be understood that system, method and kit embodiments of the present invention will inherently require a source of power, such as access to utility power. Contemplated system and kit embodiments may further include a portable generator as a source for powering the equipment. Still other embodiments contemplate the inclusion of a battery sufficient to run all of the equipment for added remote capability where a power outlet or generator are not available. Such generators and battery equipment are within the knowledge of one of ordinary skill in the art and thus will not be further elaborated herein.

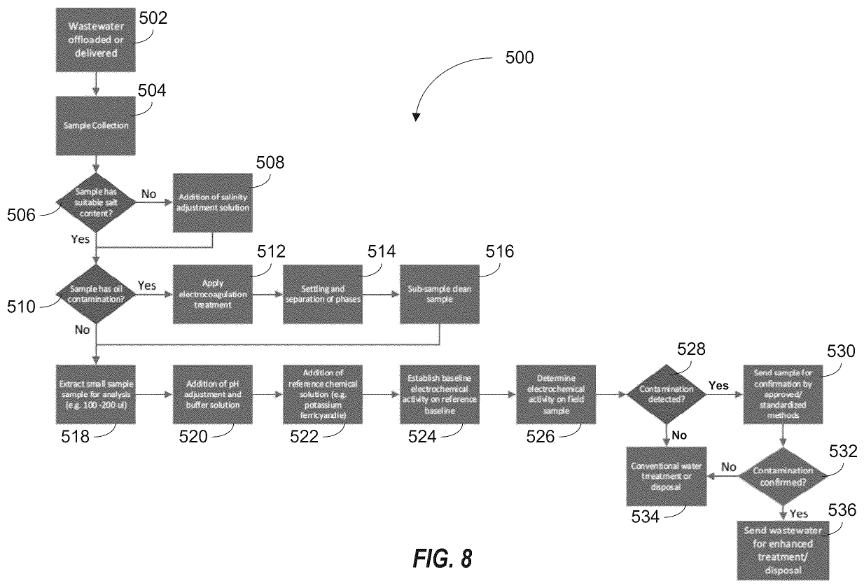

Having described various system and kit embodiments of the present invention, further method embodiments of the present invention are described below. FIG. 8 is a flowchart of an embodiment of a method 500 for detecting PFAS in wastewater, according to the present invention. The embodiment of method 500 may be used to perform low-cost detection of analytes of PFAS in a liquid medium. Embodiments of method 500 are capable of testing for PFAS in OWW and “clean” or “treated” wastewater that is not contaminated with oil or grease. According to specific embodiments of method 500, the PFAS analyte detection may be performed with a disposable, portable, and rugged system or kit. The system and kit embodiments only require a limited amount of sample solution (~microliters) to determine if an analyte is present or not.

As shown in FIG. 8, the embodiment of method 500 may include obtaining 502 the entire wastewater volume of concern by offloading or delivering the wastewater from its source, e.g., bilge compartment of a vessel, or some other wastewater source for which PFAS compound detection is desired. The embodiment of method 500 may further include collecting 504 an original sample of the wastewater for field analysis. This collecting step 504 may be facilitated by a liquid storage container as described herein. The embodiment of method 500 may further include determining 506 whether the original sample of the wastewater has suitable salt content to perform the analysis. If not, the embodiment of method 500 may further include adding salinity 508 to the original wastewater sample to reach a suitable salt content for performing the PFAS detection. The addition 508 of salt may be perform as described elsewhere herein.

If the original sample of the wastewater was found to have proper salinity 506 or has been adjusted to have to proper salinity 508, the embodiment of method 500 may further include determining 510 if the proper salinity sample has oil or grease contamination. This determination 510 is typically a visual observation for a surface sheen on the proper salinity sample or visual observation for turbidity from emulsions. If the proper salinity sample is contaminated with oil or grease, i.e., OWW, the embodiment of method 500 may further include applying 512 an electrocoagulation treatment to the proper salinity sample. As part of the application of the electrocoagulation treatment 512, the embodiment of method 500 may further include the settling and separating 514 of the oil or grease from OWW as a sediment layer 16 (FIG. 1) on the bottom of the proper salinity OWW and/or sludge layer 18 (FIG. 1) on the top of the proper salinity OWW. Once the electrocoagulation application 512 and the settling and separation 514 steps are completed, the embodiment of method 500 may further include sub-sampling 516 the treated OWW to obtain a clean proper salinity sample of the wastewater ready for PFAS detection. The clean proper salinity sample may be found in between the sediment layer 16 and the sludge layer 18 (see, FIG. 1). Also, a clean proper salinity sample may result from the determining step 510, if the answer is negative.

The embodiment of method 500 may further include extracting 518 a small sample portion of the clean proper salinity sample, e.g., 100-200 μL, using a pipette, preferably disposable. An optional centrifuge tube and centrifuge machine are useful here for receiving small sample portion of the clean proper salinity sample from the disposable pipette and for mixing. The embodiment of method 500 may further include adding 520 a pH adjustment and buffer solution to the clean proper salinity sample (and mixing, or preferably vortex using the centrifuge machine) to obtain a pH adjusted and buffered sample. Note that the proper salinity determination step 506 and the salinity adjustment step 508 may be moved from above and combined in step 520, according to other embodiments.

The embodiment of method 500 may further include adding 522 a reference solution with a preselected reporter chemical having well-known electrochemical characteristics, e.g., potassium ferricyanide, to the pH adjusted and buffered sample to obtain a field sample ready for analysis. The embodiment of method 500 may further include establishing 524 a baseline electrochemical activity measurement on the reference solution. The electrochemical activity measurement 524 may be performed according to the exemplary procedures described herein using an electrochemical reader also described herein to generate a reference baseline curve, see, e.g., curve 330 (FIG. 6). The embodiment of method 500 may further include determining 526 an electrochemical activity on the field sample. Step 526 may include the sub-steps of re-using the same SPE, first by removing the remaining reference solution from the SPE well and rinsing with ultrapure or DI water, then applying a preselected measure of the sample solution into the well of the SPE, inserting the sample SPE into the electrochemical reader and performing the potentiostat and voltammetry measurements described herein to obtain a sample electrochemical activity curve.

The embodiment of method 500 may further include determining 528 if the sample solution is contaminated with PFAS. If the sample has PFAS contamination, its activity may appear as curve 340 (FIG. 6), or with some other relative reduced peak height relative to the reference baseline, see, e.g., arrow 350 (FIG. 6) reflecting the presence of PFAS and relative concentration of PFAS in the sample solution which can be inferred from the relative reduction. According to the illustrated embodiment, method 500 may further include sending 530 the PFAS positive sample to a certified laboratory using approved or standardized procedures for confirmation of PFAS contamination, if “yes” indicated at step 528. According to the illustrated embodiment, method 500 may further include confirming 532 the PFAS contamination indicated at step 528 at an approved or standardized laboratory. According to the embodiment of method 500 illustrated in FIG. 8, if PFAS contamination is not detected at step 528 or confirmed at step 532, then method 500 may further include treating or disposing 534 of the entire offloaded wastewater volume 502 using conventional procedures, e.g., wastewater treatment shore-side (in port) or discharged per regulation. The embodiment of method 500 may further include sending 536 the entire offloaded wastewater volume 502 which has been confirmed as PFAS positive at step 532 shore-side for enhanced treatment or disposal.

Having described particular embodiments of the present invention with reference to the drawings, additional generic embodiments of the invention are now described. An embodiment of a system for detecting PFAS in wastewater is disclosed. The system embodiment may include an optional sample collection container for storing a volume of the wastewater and from which a sample of the wastewater may be obtained. The system embodiment may further include a salinity subsystem configured for measuring and adjusting salinity of the sample wastewater to obtain a proper salinity sample of the wastewater. The system embodiment may further include an optional electrocoagulation subsystem configured for cleaning oil and grease contaminants, if necessary, from the proper salinity sample of the wastewater to obtain a clean proper salinity sample of the wastewater. The system embodiment may further include a PFAS detection subsystem configured for detecting concentration of the PFAS in the clean proper salinity sample of the wastewater and reporting same to a user.

According to another system embodiment, the optional electrocoagulation subsystem may further include an electrocoagulation electrode assembly for placement at least partially within the proper salinity sample of the wastewater. According to this system embodiment, the optional electrocoagulation subsystem may further include a power supply for delivering a preselected voltage and for a preselected time across the electrocoagulation electrode assembly at least partially submerged within the proper salinity sample of the wastewater to obtain a clean proper salinity sample of the wastewater. According to particular embodiments of the system, the electrocoagulation electrode assembly may include at least one anode separated from at least one cathode by at least one insulating spacer. According to still more particular embodiments of the system, the at least one anode and the at least one cathode may be formed aluminum. According to yet another system embodiment of the optional electrocoagulation subsystem, the oil and grease contaminants settle to a bottom sediment layer and/or floats to a top sludge layer of the proper salinity sample during electrocoagulation as fractions that may be removed from the proper salinity sample to obtain the clean proper salinity sample.

According to one system embodiment, the PFAS detection subsystem may include an optional pH adjustment and buffer solution for selectively adding to the clean proper salinity sample of the wastewater, if necessary, to obtain a field sample ready for analysis. According to this system embodiment, the PFAS detection subsystem may further include a reference solution selected for generating a reference baseline voltammetry measurement. According to this system embodiment, the PFAS detection subsystem may further include electrochemical sensor electrodes configured with a preselected sensitivity to PFAS and configured for contact with the reference solution initially and then subsequently with a combination of the reference solution and the field sample. According to this system embodiment, the PFAS detection subsystem may further include an electrochemical reader in electrical communication with the electrochemical sensor electrodes and configured for generating the reference baseline voltammetry response when the electrochemical sensor electrodes are in contact with the reference solution, and configured for generating a PFAS voltammetry response representative of the PFAS within the field sample when the electrochemical sensor electrodes are in contact with a mixture of the field sample added to the reference solution. According to this system embodiment, the PFAS detection subsystem may further include a processor in communication with the electrochemical reader and configured to store and compare the PFAS voltammetry response to the baseline voltammetry response and thereby determine the concentration of the PFAS in the field sample and report same to a user.

According to still another system embodiment of the PFAS detection subsystem, the PFAS may include PFOS. According to various system embodiments of the PFAS detection subsystem, the reference solution may be a solution of potassium ferrocyanide, potassium ferricyanide, ruthenium hexamine, methyl viologen, or copper sulfate. According to still yet another system embodiment of the PFAS detection subsystem, the electrochemical sensor electrodes may further include screen-printed electrodes (SPEs) formed on a substrate using selected screen-printed conductive inks. According to particular system embodiments of the PFAS detection subsystem, each of the selected screen-printed conductive inks may be formed of silver, carbon, gold, platinum, ruthenium oxide, aluminum, cobalt, chromium, indium tin oxide, molybdenum, lead, palladium and tungsten. According to various additional system embodiments of the PFAS detection subsystem, the electrochemical sensor electrodes may further include a surface roughness, altered chemical properties and/or an altered geometry to selectively change the amount of absorption of the PFAS on the electrochemical sensor electrodes. According to one system embodiment of the PFAS detection subsystem, the electrochemical sensor electrodes may further include a well for receiving a given solution for analysis and a connector for insertion into a socket of the electrochemical reader.

An embodiment of a method for field testing wastewater to detect PFAS is disclosed. The method embodiment may include providing a system for detecting the PFAS in wastewater. The method embodiment may further include obtaining the wastewater from its source. The method embodiment may further include collecting a sample of the wastewater for field analysis. The method embodiment may further include determining whether the sample has suitable salt content. If the sample does not have suitable salinity, then adjusting salinity of the sample to a proper salinity content by adding a salinity adjustment solution to the sample to obtain a proper salinity sample. If the sample does have suitable salinity, then continue with the proper salinity sample. The method embodiment may further include determining whether the proper salinity sample has oil or grease contamination. If the proper salinity sample does have oil or grease contamination, then applying an electrocoagulation treatment, settling and separating an oil contamination portion of the proper salinity sample, and continuing with a remaining portion of a clean proper salinity sample. If the proper salinity sample does not have oil or grease contamination, then continue with the clean proper salinity sample. The method embodiment may further include extracting a preselected small volume sample from the clean proper salinity sample. The method embodiment may further include adding a pH adjustment and buffer solution to the clean proper salinity small volume sample to obtain a field sample ready for analysis. The method embodiment may further include preparing a reference chemical solution to obtain a reference sample. The method embodiment may further include establishing a baseline electrochemical activity on the reference sample. The method embodiment may further include determining a field sample electrochemical activity on the field sample. The method embodiment may further include comparing the field sample electrochemical activity to the baseline electrochemical activity to determine if the field sample is contaminated with PFAS according to the field testing.

According to another embodiment, the method may further include sending the wastewater sample to a standard testing laboratory for confirmation of the PFAS positive field test if the field sample is determined to be contaminated with PFAS, PFAS positive, according to the field testing. If the PFAS positive field test is confirmed by the standard testing laboratory, then the method embodiment may further include sending all wastewater for enhanced treatment or disposal. Alternatively, if the PFAS positive field test is not confirmed by the standard testing laboratory, then sending all wastewater for conventional water treatment or disposal.

According to one embodiment of the method, the system provided may further include a sample collection container for storing a volume of the wastewater and from which a sample of the wastewater may be obtained. According to this embodiment of the method, the system provided may further include a salinity subsystem configured for measuring and adjusting salinity of the sample wastewater to obtain a proper salinity sample of the wastewater. According to this embodiment of the method, the system provided may further include an optional electrocoagulation subsystem configured for cleaning oil and grease contaminants from a proper salinity sample of oily wastewater, if necessary, to obtain a clean proper salinity sample of the wastewater. According to this embodiment of the method, the system provided may further include a PFAS detection subsystem configured for detecting presence and concentration of the PFAS in the clean proper salinity sample of the wastewater.

According to one embodiment of the method, the electrocoagulation subsystem may further include an electrocoagulation electrode for placement at least partially within the proper salinity sample of the wastewater. According to this method embodiment, the electrocoagulation subsystem may further include a power supply for delivering a preselected voltage and for a preselected time to the electrocoagulation electrode submerged at least partially within the proper salinity sample of the wastewater to obtain a clean proper salinity sample of the wastewater. According to another embodiment of the method, the electrocoagulation electrode assembly may include at least one anode separated from at least one cathode by at least one insulating spacer. According to still another embodiment of the method, the at least one anode and the at least one cathode may be formed of aluminum. According to another method embodiment, the oil and grease contamination in the oily wastewater settles to a bottom and/or floats to a top of the proper salinity sample during electrocoagulation as fractions that may be removed from the proper salinity sample to obtain the clean proper salinity sample.

According to still another embodiment of the method, the PFAS detection subsystem may include an optional pH adjustment and buffer solution for selectively adding to the clean proper salinity sample of the wastewater, if necessary, to obtain a field sample ready for analysis. According to this embodiment of the method, the PFAS detection subsystem may further include a reference solution selected for generating a reference baseline voltammetry measurement. According to this embodiment of the method, the PFAS detection subsystem may further include electrochemical sensor electrodes configured with a preselected sensitivity to PFAS and configured for contact with the reference solution initially and then subsequently with a combination of the reference solution and the field sample. According to this embodiment of the method, the PFAS detection subsystem may further include an electrochemical reader in electrical communication with the electrochemical sensor electrodes and configured for generating the reference baseline voltammetry response when the electrochemical sensor electrodes are in contact with the reference solution, and configured for generating a field sample voltammetry response representative of the PFAS within the field sample when the electrochemical sensor electrodes are in contact with a mixture of the field sample added to the reference solution. According to this embodiment of the method, the PFAS detection subsystem may further include a processor in communication with the electrochemical reader and configured to store and compare the field sample voltammetry response to the baseline voltammetry response and thereby determine the concentration of the PFAS in the field sample and report same to a user.

According to a particular embodiment of the method, the PFAS may include PFOS. According to another embodiment of the system, the reference solution of the PFAS detection subsystem may include one of the following: potassium ferrocyanide, potassium ferricyanide, ruthenium hexamine, methyl viologen and copper sulfate. According to another embodiment of the method, the electrochemical sensor electrodes may further include screen-printed electrodes (SPEs) formed on a substrate using selected screen-printed conductive inks. According to particular embodiments of the method, each of the selected screen-printed conductive inks may be formed of one of the following materials: silver, carbon, gold, platinum, ruthenium oxide, aluminum, cobalt, chromium, indium tin oxide, molybdenum, lead, palladium and tungsten.

An embodiment of a field kit for detecting PFAS in wastewater is disclosed. The field kit embodiment may include a storage and transportation container for storing and transporting the field kit. The field kit embodiment may further include a sample collection container for storing a volume of the wastewater and from which a sample of the wastewater may be obtained. The field kit embodiment may further include a salinity subsystem configured for measuring and adjusting salinity of the sample wastewater to obtain a proper salinity sample of the wastewater. The field kit embodiment may further include an optional electrocoagulation subsystem configured for cleaning oil and/or grease from the proper salinity sample of the wastewater, if necessary, to obtain a clean proper salinity sample of the wastewater. The field kit embodiment may further include a PFAS detection subsystem configured for detecting presence and concentration of the PFAS in the clean proper salinity sample of the wastewater.

According to another embodiment of a field kit for detecting PFAS in wastewater, the optional electrocoagulation subsystem may further include an electrocoagulation electrode for placement at least partially within the proper salinity sample of the wastewater. According to this embodiment of a field kit, the optional electrocoagulation subsystem may further include a power supply for delivering a preselected voltage and for a preselected time to the electrocoagulation electrode to obtain a clean proper salinity sample of the wastewater. According to yet another embodiment of a field kit for detecting PFAS in wastewater, the electrocoagulation electrode assembly may further include at least one anode separated from at least one cathode by at least one insulating spacer. According to a particular embodiment of a field kit for detecting PFAS in wastewater, the at least one anode and the at least one cathode may be formed of aluminum. According to another embodiment of a field kit for detecting PFAS in wastewater, the oil and/or grease settles to a bottom and/or floats to a top of the proper salinity sample during electrocoagulation as fractions that may be removed from the proper salinity sample to obtain the clean proper salinity sample.

According to yet another embodiment of a field kit for detecting PFAS in wastewater, the PFAS detection subsystem may further include an optional pH adjustment and buffer solution for selectively adding to the clean proper salinity sample of the wastewater, if necessary, to obtain a field sample ready for analysis. According to this embodiment of a field kit for detecting PFAS in wastewater, the PFAS detection subsystem may further include a reference solution selected for generating a reference baseline voltammetry measurement. According to this embodiment of a field kit for detecting PFAS in wastewater, the PFAS detection subsystem may further include electrochemical sensor electrodes configured with a preselected sensitivity to PFAS and configured for contact with the reference solution initially and then subsequently with a combination of the reference solution and the field sample. According to this embodiment of a field kit for detecting PFAS in wastewater, the PFAS detection subsystem may further include an electrochemical reader in electrical communication with the electrochemical sensor electrodes and configured for generating the reference baseline voltammetry response when the electrochemical sensor electrodes are in contact with the reference solution, and configured for generating a field sample voltammetry response representative of the PFAS within the field sample when the electrochemical sensor electrodes are in contact with a mixture of the field sample added to the reference solution. According to this embodiment of a field kit for detecting PFAS in wastewater, the PFAS detection subsystem may further include a processor in communication with the electrochemical reader and configured to store and compare the field sample voltammetry response to the baseline voltammetry response and thereby determine the concentration of the PFAS in the field sample and report same to a user.

According to a particular embodiment of a field kit for detecting PFAS in wastewater, the PFAS may include PFOS. According to various embodiments of a field kit for detecting PFAS in wastewater, the reference solution may include one of: potassium ferrocyanide, potassium ferricyanide, ruthenium hexamine, methyl viologen and copper sulfate. According to one embodiment of a field kit for detecting PFAS in wastewater, the electrochemical sensor electrodes may further include screen-printed electrodes (SPEs) formed on a substrate using selected screen-printed conductive inks. According to still further embodiments of a field kit for detecting PFAS in wastewater, each of the selected screen-printed conductive inks may include one of the following materials: silver, carbon, gold, platinum, ruthenium oxide, aluminum, cobalt, chromium, indium tin oxide, molybdenum, lead, palladium and tungsten.

In understanding the scope of the present invention, the term “configured” as used herein to describe a component, section or part of a device includes hardware and/or software that is constructed and/or programmed to carry out the desired function. In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed.

From the above description of the embodiments of a system, method and kit for detecting PFAS, it is manifest that various alternative structures, components and method steps may be used for implementing features of the present invention without departing from the scope of the claims. The described embodiments are to be considered in all respects as illustrative and not restrictive. It will further be understood that the present invention may suitably comprise, consist of, or consist essentially of the component parts, method steps and limitations disclosed herein. The method and/or apparatus disclosed herein may be practiced in the absence of any element that is not specifically claimed and/or disclosed herein.

While the foregoing advantages of the present invention are manifested in the detailed description and illustrated embodiments of the invention, a variety of changes can be made to the configuration, design and construction of the invention to achieve those advantages. Hence, reference herein to specific details of the structure and function of the present invention is by way of example only and not by way of limitation.

What is claimed is:

1. A system for detecting per- and polyfluorinated compounds (PFAS) in wastewater, the system comprising:
an optional sample collection container for storing a volume of the wastewater and from which a sample of the wastewater may be obtained;
a salinity subsystem configured for measuring and adjusting salinity of the sample wastewater to obtain a proper salinity sample of the wastewater;
an optional electrocoagulation subsystem configured for cleaning oil and grease contaminants, if necessary, from the proper salinity sample of the wastewater to obtain a clean proper salinity sample of the wastewater; and
a PFAS detection subsystem configured for detecting concentration of the PFAS in the clean proper salinity sample of the wastewater and reporting same to a user.

2. The system according to claim 1, wherein the optional electrocoagulation subsystem comprises:
an electrocoagulation electrode assembly for placement at least partially within the proper salinity sample of the wastewater; and
a power supply for delivering a preselected voltage and for a preselected time across the electrocoagulation electrode assembly at least partially submerged within the proper salinity sample of the wastewater to obtain a clean proper salinity sample of the wastewater.

3. The system according to claim 2, wherein the electrocoagulation electrode assembly comprises at least one anode separated from at least one cathode by at least one insulating spacer.

4. The system according to claim 3, wherein the at least one anode and the at least one cathode comprise aluminum.

5. The system according to claim 1, wherein the oil and grease contaminants settle to a bottom sediment layer and/or floats to a top sludge layer of the proper salinity sample during electrocoagulation as fractions that may be removed from the proper salinity sample to obtain the clean proper salinity sample.

6. The system according to claim 1, wherein the PFAS detection subsystem comprises:
an optional pH adjustment and buffer solution for selectively adding to the clean proper salinity sample of the wastewater, if necessary, to obtain a field sample ready for analysis;
a reference solution selected for generating a reference baseline voltammetry measurement;
electrochemical sensor electrodes, each configured with a preselected sensitivity to PFAS and configured for contact with the reference solution initially and then subsequently with a combination of the reference solution and the field sample;
an electrochemical reader in electrical communication with the electrochemical sensor electrodes and configured for generating the reference baseline voltammetry response when the electrochemical sensor electrodes are in contact with the reference solution, and configured for generating a field sample voltammetry response representative of the PFAS within the field sample when the electrochemical sensor electrodes are in contact with a mixture of the field sample added to the reference solution; and
a processor in communication with the electrochemical reader and configured to store and compare the field sample voltammetry response to the baseline voltammetry response and thereby determine the concentration of the PFAS in the field sample and report same to a user.

7. The system according to claim 1, wherein the PFAS comprise perfluorooctanesulfonic acid (PFOS).

8. The system according to claim 6, wherein the reference solution is selected from the group consisting of: potassium ferrocyanide, potassium ferricyanide, ruthenium hexamine, methyl viologen and copper sulfate.

9. The system according to claim 6, wherein the electrochemical sensor electrodes further comprises screen-printed electrodes (SPEs) formed on a substrate using selected screen-printed conductive inks.

10. The system according to claim 9, wherein each of the selected screen-printed conductive inks are selected from the group consisting of: silver, carbon, gold, platinum, ruthenium oxide, aluminum, cobalt, chromium, indium tin oxide, molybdenum, lead, palladium and tungsten.

11. The system according to claim 9, wherein a surface roughness and/or geometry, and/or chemical properties of the electrochemical sensor electrodes is altered to change the amount of absorption of the PFAS on the electrochemical sensor electrodes.

12. The system according to claim 6, wherein the electrochemical sensor electrodes further comprise a well for receiving a given solution for analysis and a connector for insertion into a socket of the electrochemical reader.

13. A method for field testing wastewater to detect per- and polyfluorinated compounds (PFAS), the method comprising:
providing a system for detecting the PFAS in wastewater;
obtaining the wastewater from its source;
collecting a sample of the wastewater for field analysis;
determining whether the sample has suitable salt content;
if no, then adjusting salinity of the sample to a proper salinity content by adding salinity adjustment solution to the sample to obtain a proper salinity sample; and
if yes, then continuing with the proper salinity sample;
determining whether the proper salinity sample has oil or grease contamination;
if yes, then:
applying an electrocoagulation treatment;
settling and separating an oil contamination portion of the proper salinity sample; and
continuing with a remaining portion of a clean proper salinity sample;
if no, then continuing with the clean proper salinity sample;
extracting a small volume sample from the clean proper salinity sample;
adding a pH adjustment and buffer solution to the clean proper salinity small volume sample to obtain a field sample ready for analysis;
preparing a reference chemical solution to obtain a reference sample;
establishing a baseline electrochemical activity on the reference sample;
determining a field sample electrochemical activity on the field sample; and
comparing the field sample electrochemical activity to the baseline electrochemical activity to determine if the field sample is contaminated with PFAS according to the method for field testing.

14. The method according to claim 13, further comprising:
if the field sample is determined to be contaminated with PFAS, i.e., PFAS positive, according to the method for field testing, then sending the wastewater sample to a standard testing laboratory for confirmation of the PFAS positive field test; and
if the PFAS positive field test is confirmed by the standard testing laboratory, then sending all wastewater for enhanced treatment or disposal; or
if the PFAS positive field test is not confirmed by the standard testing laboratory, then sending all wastewater for conventional water treatment or disposal.

15. The method according to claim 13, wherein the system provided further comprises:
a sample collection container for storing a volume of the wastewater and from which a sample of the wastewater may be obtained;
a salinity subsystem configured for measuring and adjusting salinity of the sample wastewater to obtain a proper salinity sample of the wastewater;
an optional electrocoagulation subsystem configured for cleaning oil and grease contaminants from a proper salinity sample of oily wastewater, if necessary, to obtain a clean proper salinity sample of the wastewater; and
a PFAS detection subsystem configured for detecting presence and concentration of the PFAS in the clean proper salinity sample of the wastewater.

16. The method according to claim 15, wherein the optional electrocoagulation subsystem further comprises:
an electrocoagulation electrode assembly configured for placement at least partially within the proper salinity sample of the wastewater; and
a power supply for delivering a preselected voltage and for a preselected time across the electrocoagulation electrode assembly submerged at least partially within the proper salinity sample of the wastewater to obtain a clean proper salinity sample of the wastewater.

17. The method according to claim 16, wherein the electrocoagulation electrode assembly comprises at least one anode separated from at least one cathode by at least one insulating spacer.

18. The method according to claim 17, wherein the at least one anode and the at least one cathode comprise aluminum.

19. The method according to claim 15, wherein the oil and grease contaminants in the oily wastewater settles to a bottom and/or floats to a top of the proper salinity sample during electrocoagulation as fractions that may be removed from the proper salinity sample to obtain the clean proper salinity sample.

20. The method according to claim 15, wherein the PFAS detection subsystem comprises:
an optional pH adjustment and buffer solution for selectively adding to the clean proper salinity sample of the wastewater, if necessary, to obtain a field sample ready for analysis;
a reference solution selected for generating a reference baseline voltammetry measurement;
electrochemical sensor electrodes configured with a preselected sensitivity to PFAS and configured for contact with the reference solution initially and then subsequently with a combination of the reference solution and the field sample;
an electrochemical reader in electrical communication with the electrochemical sensor electrodes and configured for generating the reference baseline voltammetry response when the electrochemical sensor electrodes are in contact with the reference solution, and configured for generating a field sample voltammetry response representative of the PFAS within the field sample when the electrochemical sensor electrodes are in contact with a mixture of the field sample added to the reference solution; and
a processor in communication with the electrochemical reader and configured to store and compare the field sample voltammetry response to the baseline voltammetry response and thereby determine the concentration of the PFAS in the field sample and report same to a user.

21. The method according to claim 13, wherein the PFAS comprise perfluorooctanesulfonic acid (PFOS).

22. The method according to claim 20, wherein the reference solution is selected from the group consisting of: potassium ferrocyanide, potassium ferricyanide, ruthenium hexamine, methyl viologen and copper sulfate.

23. The method according to claim 20, wherein the electrochemical sensor electrodes further comprise screen-printed electrodes (SPEs) formed on a substrate using selected screen-printed conductive inks.

24. The method according to claim 23, wherein each of the selected screen-printed conductive inks are selected from the group consisting of: silver, carbon, gold, platinum, ruthenium oxide, aluminum, cobalt, chromium, indium tin oxide, molybdenum, lead, palladium and tungsten.

25. A field kit for detecting per- and polyfluorinated compounds (PFAS) in wastewater, the field kit comprising:
a storage and transportation container for storing and transporting the field kit;
a sample collection container for storing a volume of the wastewater and from which a sample of the wastewater may be obtained;
a salinity subsystem configured for measuring and adjusting salinity of the sample wastewater to obtain a proper salinity sample of the wastewater;
an optional electrocoagulation subsystem configured for cleaning oil and/or grease from the proper salinity sample of the wastewater, if necessary, to obtain a clean proper salinity sample of the wastewater; and
a PFAS detection subsystem configured for detecting presence and concentration of the PFAS in the clean proper salinity sample of the wastewater.

26. The field kit according to claim 25, wherein the optional electrocoagulation subsystem comprises:
an electrocoagulation electrode assembly for placement at least partially within the proper salinity sample of the wastewater; and
a power supply for delivering a preselected voltage and for a preselected time across the electrocoagulation electrode to obtain a clean proper salinity sample of the wastewater.

27. The field kit according to claim 26, wherein the electrocoagulation electrode assembly comprises at least one anode separated from at least one cathode by at least one insulating spacer.

28. The field kit according to claim 27, wherein the at least one anode and the at least one cathode comprise aluminum.

29. The field kit according to claim 25, wherein the oil and/or grease settles to a bottom and/or floats to a top of the proper salinity sample during electrocoagulation as fractions that may be removed from the proper salinity sample to obtain the clean proper salinity sample.

30. The field kit according to claim 25, wherein the PFAS detection subsystem comprises:
an optional pH adjustment and buffer solution for selectively adding to the clean proper salinity sample of the wastewater, if necessary, to obtain a field sample ready for analysis;
a reference solution selected for generating a reference baseline voltammetry measurement;
electrochemical sensor electrodes configured with a preselected sensitivity to PFAS and configured for contact with the reference solution initially and then subsequently with a combination of the reference solution and the field sample;
an electrochemical reader in electrical communication with the electrochemical sensor electrodes and configured for generating the reference baseline voltammetry response when the electrochemical sensor electrodes are in contact with the reference solution, and configured for generating a field sample voltammetry response representative of the PFAS within the field sample when the electrochemical sensor electrodes are in contact with a mixture of the field sample added to the reference solution; and
a processor in communication with the electrochemical reader and configured to store and compare the field sample voltammetry response to the baseline voltammetry response and thereby determine the concentration of the PFAS in the field sample and report same to a user.

31. The field kit according to claim 25, wherein the PFAS comprise perfluorooctanesulfonic acid (PFOS).

32. The field kit according to claim 30, wherein the reference solution is selected from the group consisting of: potassium ferrocyanide, potassium ferricyanide, ruthenium hexamine, methyl viologen and copper sulfate.

33. The field kit according to claim 30, wherein the electrochemical sensor electrodes further comprises screen-printed electrodes (SPEs) formed on a substrate using selected screen-printed conductive inks.

34. The field kit according to claim 33, wherein each of the selected screen-printed conductive inks are selected from the group consisting of: silver, carbon, gold, platinum, ruthenium oxide, aluminum, cobalt, chromium, indium tin oxide, molybdenum, lead, palladium and tungsten.
